(19) United States
(12) Patent Application Publication
(10) Pub. No.: US 2013/0338361 A1
(43) Pub. Date: Dec. 19, 2013

(54) RADIOFLUORINATION METHOD

(71) Applicants: Helen May Betts, Amersham (GB); Imtiaz Khan, Amersham (GB); Edward George Robins, Singapore (SG)

(72) Inventors: Helen May Betts, Amersham (GB); Imtiaz Khan, Amersham (GB); Edward George Robins, Singapore (SG)

(73) Assignee: GE HEALTHCARE LIMITED, LITTLE CHALFONT, BUCKINGHAMSHIRE (GB)

(21) Appl. No.: 14/001,952

(22) PCT Filed: Mar. 1, 2012
(86) PCT No.: PCT/EP2012/053547

§ 371 Date: Aug. 28, 2013

Related U.S. Application Data
(60) Provisional application No. 61/447,724, filed on Mar. 1, 2011.

(30) Foreign Application Priority Data
Mar. 1, 2011 (GB) 1103455.0

(51) Int. Cl. C07B 59/00; C07D 295/135; C07D 213/81; C07D 213/61

(52) U.S. Cl. CPC C07B 59/002; C07D 213/61; C07D 295/135; C07D 213/81
USPC 544/360; 546/345; 546/323; 422/159

(57) ABSTRACT

Provided by the present invention is a novel method for obtaining an ¹⁸F-labelled compound wherein said compound comprises an ¹⁸F-labelled pyridyl ring. The method of the invention is advantageous over the prior art methods as it provides these compounds in higher radiochemical yields than have been possible with previous methods. Also provided by the present invention is an ¹⁸F-labelled synthon useful in the method of the invention.

### TECHNICAL FIELD OF THE INVENTION

The present invention relates to a method for radiosynthesis and more specifically a novel method for the synthesis of ¹⁸F-labelled compounds. The invention also relates to a novel synthon for use in the inventive method of synthesis.

### DESCRIPTION OF RELATED ART

In order to expand the range of applications for positron emission tomography (PET) there is an interest in developing synthetic methods for new PET tracers, i.e. biologically useful compounds labelled with ¹¹C, ¹⁸F or ⁷⁶Br. Currently, the most widely-used of these radiotracers for PET imaging is ¹⁸F.

Typically, the synthesis of a PET tracer including its purification should be completed within three half-lives of the radiotracer. ¹⁸F has a relatively short half-life of 109.7 minutes and as such methods for its incorporation into a PET tracer demands fast and high-yielding reactions that can be performed on a small scale and under mild conditions.

Direct labelling is desirable as it introduces ¹⁸F at the last possible step. However, direct labelling tends only to be possible using [¹⁸F]fluoride in a nucleophilic substitution reaction can require the presence of activating groups, proton-free conditions and typically high temperatures of above 100° C. The reader is referred to Coenen (“PET Chemistry: The Driving Force in Molecular Imaging”, Ernst Schering Research Foundation Workshop 62, Schubiger et al, Eds; Springer 2007 pp 15-50) for more detail on typical direct labelling reaction conditions.

Alternatively, ¹⁸F can be introduced as part of a synthon. With this approach, an activated precursor is radiofluorinated, and used in subsequent reactions to prepare the desired radiofluorinated product. Many classes of synthons are known for the introduction of an ¹⁸F-labelled aromatic group, e.g. [¹⁸F]fluorobenzaldehydes, [¹⁸F]fluoroarylketones, [¹⁸F]fluorobenzoic acid, [¹⁸F]fluoronitrobenzene, [¹⁸F]fluorobenzonitrile, [¹⁸F]fluorosulfonyl arenes, and [¹⁸F]fluorohalobenzenes. These classes of synthons, methods to obtain them, and how they can be converted into PET tracers are described in a review by Ermert and Coenen (2010 Current Radiopharmaceuticals; 3: 127-160).

¹⁸F-labelled fluoropyridines have found increasing application in PET imaging, and strategies to obtain these compounds are gaining increasing attention. A review by Dolle (2005 Curr Pharm Des; 11: 3221-3235) describes how a variety of [¹⁸F]fluoropyridyl-containing compounds can be obtained by nucleophilic heteroaromatic substitution at the ortho position with [¹⁸F]fluoride. A specific example of this labelling strategy is reported by Roger et al (2006 J Label Comp Radiopharm; 49: 489-504), who describe the synthesis of 2-exo-(2′-[¹⁸F]fluoro-3′-(4-fluorophenyl)-pyridin-5′-yl)-7-azabicyclo[2.2.1]heptane by nucleophilic aromatic substitution of a precursor compound as follows:

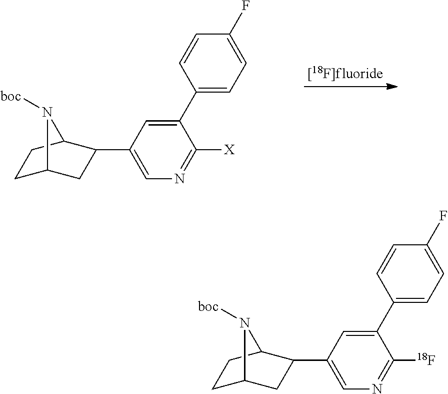

wherein X in the scheme represents Cl or Br, with overall radiochemical yields reported as 8-9%. 4-[¹⁸F]fluoropyridyl derivatives can also be obtained using such an approach, but not feasibly for 3-[¹⁸F]fluoropyridyl derivatives where very strongly electron-withdrawing groups would need to be present, and even then the reaction would likely be low-yielding.

Abrahim et al (2006 J Label Comp Radiopharm; 49: 345-356) report the synthesis of 5-[¹⁸F]fluoro-2-pyridinamine and 6-[¹⁸F]fluoro-2-pyridinamine. In this approach a carbonyl was used para to a bromine leaving group to obtain the para radiofluorinated intermediate in 20-30% radiochemical yields as follows:

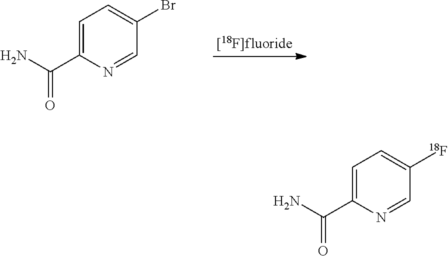 [SMILES: NC(=O)c1ccc(Br)cn1 ; NC(=O)c1ccc([18F])cn1]

In the initial attempts to obtain the 5-[¹⁸F]fluoro-2-pyridinamine synthon using a nitro starting compound, Abrahim reported obtaining 5-bromo-2-[¹⁸F]fluoropyridine as an unwanted side-product and consequently abandoned this approach.

LaBeaume et al (2010 Tet Letts; 51: 1906-1909) describe microwave-assisted methods for direct fluorination of nitro intermediates to obtain fluorinated compounds. A variety of nitro substrates were fluorinated using the methods described, including 2-bromo-6-nitropyridine, which was fluorinated with an excess of tetrabutylammonium fluoride (TBAF), yielding >95% 2-bromo-6-fluoropyridine. LaBeaume highlights that as the method gives good to excellent yields in less than 10 minutes, it is practical for use in the preparation of ¹⁸F-labelled ligands for PET imaging. LaBeaume notes that where conventional heating was tried in place of microwave heating the conversion to fluorinated product took up to −4 hours, which would clearly be unsuitable for the successful production of an ¹⁸F-labelled compound.

As a further alternative, Carroll et al (2007 J Label Comp Radiopharm; 50: 452-454) suggested diaryliodonium salts as a more generic route to obtain 3-fluoropyridines as this approach has been shown to place little or no restriction on the aromatic substituents, allowing it be used much later in the synthetic sequence as compared with the earlier-reported techniques. Radiochemical yields of 55-63% for 3-[¹⁸F]fluoropyridine were reported in this paper.

In addition various reports have discussed ¹⁸F-labelled synthons for use in obtaining ¹⁸F-labelled macromolecules. These are illustrated below:

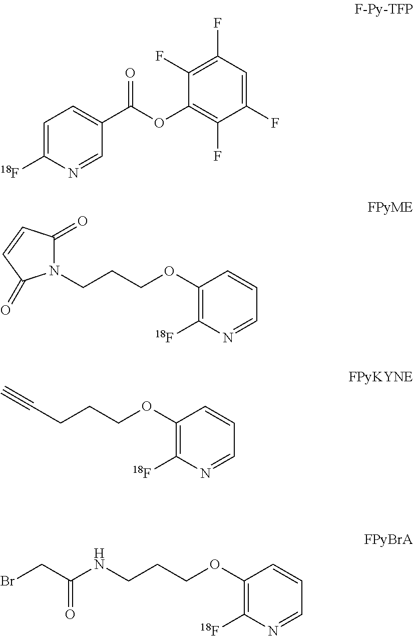 [SMILES: O=C(Oc1c(F)c(F)cc(F)c1F)c1ccc([18F])nc1 ; O=C1C=CC(=O)N1CCCOc1cccnc1[18F] ; C#CCCCOc1cccnc1[18F] ; O=C(CBr)NCCCOc1cccnc1[18F]]

Olberg et al (2010 J Med Chem; 53: 1732) report the use of F-Py-TFP (6-[¹⁸F]fluoronicotinic acid 2,3,5,6-tetrafluorophenyl ester) for peptide coupling reactions. Dolle et al (2003 J Label Comp Radiopharm; 46: S15) report the use of FPyME ([¹⁸F]fluoropyridine maleimide) for linking to thiol groups, in particular on peptides. Kuhnast et al (2008 J Label Comp Radiopharm; 51: 336) describe FPyKYNE (2-[¹⁸F]Fluoro-3-pent-4-ynyloxy-pyridine) for use in click reactions with macromolecules. Kuhnast et al (2004 Bioconj Chem; 15: 617) describe the design and use of FPyBrA (2-bromo-N-[3-(2-[¹⁸F]fluoropyridin-3-yloxy)propyl]acetamide), a [¹⁸F]fluoropyridine based halo-acetamide reagent for the labelling of oligonucleotides. Each of these synthons is useful for obtaining ¹⁸F-labelled macromolecules, but due to their relative complexity may change the physicochemical properties of a small molecule if used to add ¹⁸F.

Alternative means to obtain synthons useful in the synthesis of a broader range of ¹⁸F-labelled pyridine-containing compounds would be desirable.

### SUMMARY OF THE INVENTION

Provided by the present invention is a novel method for obtaining an ¹⁸F-labelled compound wherein said compound comprises an ¹⁸F-labelled pyridyl ring. The method of the invention is advantageous over the prior art methods as it provides these compounds in higher radiochemical yields than have been possible with previous methods. Also provided by the present invention is an ¹⁸F-labelled synthon useful in the method of the invention.

### DETAILED DESCRIPTION OF THE INVENTION

In one aspect, the present invention provides a method for [¹⁸F] labelling synthesis comprising reacting a radiolabelling precursor of Formula X:

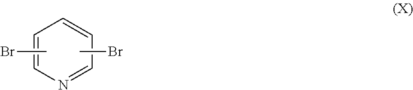

with [¹⁸F]fluoride to obtain an ¹⁸F-labelled synthon of Formula Y:

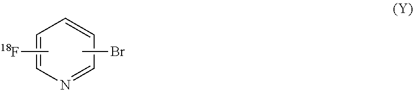

The term “synthon” refers to a constituent part of a molecule to be synthesised which is regarded as the basis of a synthetic procedure.

[¹⁸F]Fluoride used in providing the ¹⁸F-labelled synthon of Formula Y is typically obtained as an aqueous solution which is a product of the irradiation of an [¹⁸O]-water target. Various steps are carried out on the aqueous solution to convert [¹⁸F]fluoride into a reactive nucleophilic reagent, such that it is suitable for use in nucleophilic radiolabelling reactions. These steps include the elimination of water and the provision of a suitable counterion (Handbook of Radiopharmaceuticals 2003 Welch & Redvanly eds. ch. 6 pp 195-227).

Suitable counterions include large but soft metal ions such as rubidium or caesium, potassium complexed with a cryptand such as Kryptofix™, or tetraalkylammonium salts.

In a most preferred embodiment, the synthon of Formula Y is either of the following:

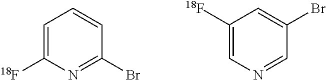 [SMILES: [18F]c1cccc(Br)n1 ; [18F]c1cncc(Br)c1]

The relevant dibromo-substituted pyridines are commercially-available. For example, 2-Bromo-6[¹⁸F]-fluoropyridine, can be readily prepared from 2,6-dibromopyridine. The present inventors have done so in 10 minutes at an end of synthesis (EOS) non-decay corrected yield of 53%.

In a preferred embodiment, the method of the present invention further comprises the step:
(ii) coupling the ¹⁸F-labelled synthon of Formula Y as defined herein with a cross-coupling partner in a transition metal-mediated coupling reaction to obtain an ¹⁸F-labelled product.

The term “cross-coupling partner” refers to a compound that can react with the synthon of Formula Y with the elimination of the synthon bromo leaving group to result in a desired ¹⁸F-labelled product. The cross-coupling partner therefore suitably comprises a chemical group that effects nucleophilic displacement of the bromo of the synthon. Non-limiting examples of such chemical groups include terminal alkene, amino, terminal alkyne, boronic acid, and organotin.

By the term “terminal alkene” is meant a double bond at the terminal end of a substituent. A preferred cross-coupling partner comprising a terminal alkene is a compound of Formula Ia as defined below.

The term “amino” refers to the group NR₂ wherein each R is hydrogen or a monovalent aliphatic or aromatic hydrocarbon substituent, as defined below. Preferably at least one R is hydrogen. A preferred cross-coupling partner comprising an amine is a compound of Formula Ie as defined below.

The term “terminal alkyne” refers to a triple bond at the terminal end of a substituent. A preferred cross-coupling partner comprising a terminal alkyne is a compound of Formula Ic as defined below.

The term “boronic acid” refers to the group —B(OH₂). A preferred cross-coupling partner comprising boronic acid is a compound of Formula Id as defined below.

The term “organotin” refers to a chemical group comprising tin and hydrocarbon substituents. Organotin compounds are also referred to as stannanes. A preferred cross-coupling partner comprising an organotin is a compound of Formula Ib as defined below.

The coupling reaction of step (ii) of the preferred embodiment of the invention is preferably site-specific and may consequently require the presence of one or more protecting groups on the cross-coupling partner. By the term “protecting group” is meant a group which inhibits or suppresses undesirable chemical reactions, but which is designed to be sufficiently reactive that it may be cleaved from the functional group in question to obtain the desired product under mild enough conditions that do not modify the rest of the molecule. Protecting groups are well known to those skilled in the art and are described in ‘Protective Groups in Organic Synthesis’, Theorodora W. Greene and Peter G. M. Wuts, (Fourth Edition, John Wiley & Sons, 2007).

The term “transition metal” includes palladium, platinum, gold, ruthenium, rhodium, and iridium. The most typically used transition metal for the coupling reactions encompassed by step (ii) of the method of the invention is palladium. Typical forms of palladium for use as a catalyst include palladium acetate, tetrakis(triphenylphosphine)palladium(0), bis(triphenylphosphine)palladium(II) dichloride, [1,1′-bis(diphenylphosphino)ferrocene]palladium(II) dichloride, and palladium on carbon (Pd/C).

Preferably, the ¹⁸F-labelled product obtained in step (ii) is a tracer suitable for PET imaging and preferably has a molecular weight <1500 Daltons; preferably <1000 Daltons. Where the ¹⁸F-labelled product is intended to be a PET tracer for imaging the central nervous system, the molecular weight is preferably <500, which is optimal for blood-brain barrier penetration.

As reported by Ermert and Coenen (2010 Current Radiopharmaceuticals; 3: 127-160), [¹⁸F]fluorohalobenzenes can be converted into a range of different target ¹⁸F-labelled molecules by means of transition metal-mediated coupling reactions, as illustrated in Scheme 1 below:

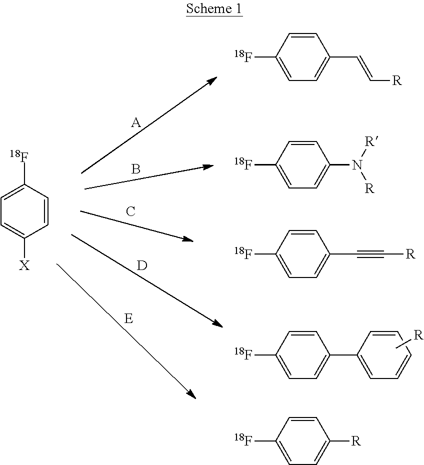

In Scheme 1, X represents bromo, chloro or iodo, and A-E represent:
(A) Heck reaction between an alkene and an aryl halides;
(B) Hartwig-Buchwald amination of an aryl halide with an amine;
(C) Sonogashira coupling between an aryl halide and an alkyne, with copper(I)iodide as a co-catalyst;
(D) Suzuki reaction between an aryl halide and boronic acid; and,
(E) Stille reaction of an organohalide and an organotin.

Each of these transition-metal catalysed reactions are well-known to the skilled person and are described e.g. in “March's Advanced Organic Chemistry: Reactions, Mechanisms, and Structures (6^{th} Edition Wiley 2007, Smith and March, Eds.); see page 792 for the Stille reaction, page 875 for Hartwig-Buchwald N-arylation, page 904 for the Sonogashira reaction and page 899 for Suzuki coupling. The [¹⁸F]-fluorobromopyridine synthon provided in step (i) of the method of the present invention can therefore be converted into a range of ¹⁸F-labelled products using these same reactions. The method of the present invention therefore allows for the synthesis of a wide range of ¹⁸F-labelled heteroaromatic PET tracers.

In one preferred embodiment of the method of the invention, said transition metal coupling reaction comprises reaction of the ¹⁸F-labelled synthon of Formula Y with a compound of Formula Ia:

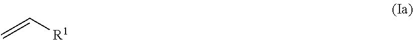

wherein R¹ is a monovalent aliphatic or aromatic hydrocarbon group;
to obtain an ¹⁸F-labelled product of Formula IIa:

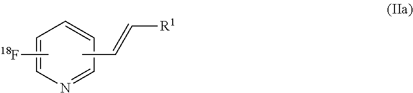

The term “monovalent aliphatic hydrocarbon group” used here and elsewhere in the specification is intended to encompass substituted or unsubstituted linear, branched or cyclic alkyl, alkenyl, or alkynyl radicals, wherein one or more of the carbons in the chain is optionally a heteroatom selected from O, S and N. The term “alkyl” refers to monovalent radical having the general formula CₙH₂ₙ₊₁, the term “alkenyl” refers to an alkyl comprising one or more double bonds, and the term “alkynyl” refers to an alkyl comprising one or more triple bonds. The term “aliphatic” relates to those parts of the radical arranged in straight or branched chains, and not containing aromatic rings.

The term “monovalent aromatic hydrocarbon group” used here and elsewhere in the specification is intended to encompass substituted or unsubstituted radicals containing one or more six-carbon rings characteristic of the benzene series and related organic groups, wherein one or more of the carbons is optionally a heteroatom selected from O, S and N.

The term also includes radicals comprising aliphatic elements in addition, wherein the aliphatic elements can be monovalent aliphatic hydrocarbon groups as defined above, or divalent derivatives thereof, provided that the designated atom's normal valency under the existing circumstances is not exceeded.

The term “substituted” as used throughout the specification means that one or more hydrogens on a designated atom is replaced with a substituent, provided that the designated atom's normal valency under the existing circumstances is not exceeded, and that the substitution results in a stable compound. Combinations of substituents and/or variables are permissible only if such combinations result in stable compounds. The term “stable compound” is meant a compound that is sufficiently robust to survive isolation to a useful degree of purity from a reaction mixture.

Non-limiting examples of “substituents” include, halo groups, hydroxy groups, oxo groups, mercapto groups, amino groups, carbamoyl groups, carboxyl groups, cyano groups, nitro groups, acyl groups, phosphate groups, sulfamyl groups, sulfonyl groups, sulfinyl groups, and combinations thereof. A substituent can also be a substituted or unsubstituted monovalent aliphatic or aromatic hydrocarbon group as defined above.

As used herein, the term “halo” or “halogen” means refers to chlorine, bromine, fluorine or iodine.

The term “oxo” refers to the group ═O.

The term “mercapto” refers to the group —SH, which is also known as thiol or sulfhydryl.

The term “carbamoyl” refers to the group —C(═O)NH₂.

The term “carboxyl” refers to the group —C(═O)OH.

The term “cyano” refers to the group —C≡N.

The term “nitro” refers to the group —NO₂.

The term “acyl” refers to the group —C(═O)-alkyl wherein alkyl is as defined above.

The term “phosphate” refers to the group —O—P(OH)₃.

The term “sulfamyl” refers to the group —S(═O)₂-amino wherein amino is as defined above.

The term “sulfonyl” refers to the group —S(═O)₂-alkyl wherein alkyl is as defined above.

The term “sulfinyl” refers to the group —S(═O)-alkyl wherein alkyl is as defined above.

In another preferred embodiment, in the method of the invention, said transition metal coupling reaction comprises reaction of the ¹⁸F-labelled synthon of Formula Y with a compound of Formula Ib:

Bu₃SnR² (Ib)
wherein Bu stands for butyl, and R² is a monovalent aliphatic or aromatic hydrocarbon group wherein both terms are as defined above;
to obtain an ¹⁸F-labelled product of Formula IIb:

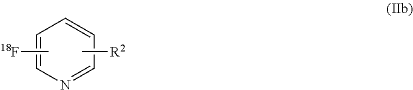

In a further preferred embodiment, in the method the invention said transition metal coupling reaction comprises reaction of the ¹⁸F-labelled synthon of Formula Y with a compound of Formula Ic:

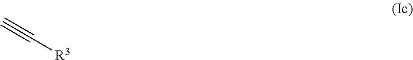

wherein R³ is a monovalent aliphatic or aromatic hydrocarbon group wherein both terms are as defined above;
to obtain an ¹⁸F-labelled product of Formula IIc:

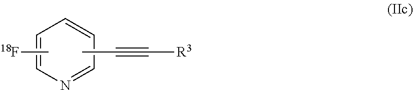

In another further preferred embodiment, in the method the invention said transition metal coupling reaction comprises reaction of the ¹⁸F-labelled synthon of Formula Y with a compound of Formula Id:

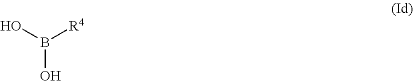

wherein R⁴ is a monovalent aliphatic or aromatic hydrocarbon group wherein both terms are as defined above;
to obtain an ¹⁸F-labelled product of Formula IId:

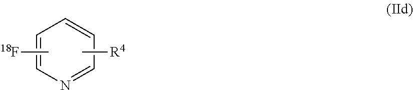

wherein R⁴ is as defined for Formula Id.

Example 4 describes such a reaction.

In a yet further preferred embodiment, in the method the invention said transition metal coupling reaction comprises reaction of the ¹⁸F-labelled synthon of Formula Y with a compound of Formula Ie:

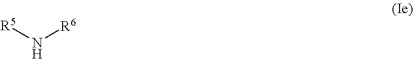

wherein R⁵ and R⁶ are independently hydrogen or a monovalent aliphatic or aromatic hydrocarbon group, wherein both terms are as defined above, or together with the nitrogen to which they are attached form a nitrogen-containing aliphatic or aromatic ring;
to obtain an ¹⁸F-labelled product of Formula IIe:

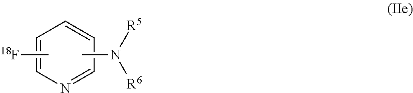

Example 2 describes such a reaction.

In the case of each of the ¹⁸F-labelled products of Formulas IIa-IIe, the suitable and preferred positions for ¹⁸F and for each substituent are as defined respectively for ¹⁸F and 8r in the synthon of Formula Y.

The term “nitrogen-containing aliphatic or aromatic ring” refers to any substituted or unsubstituted cyclic substituent that comprises at least one nitrogen heteroatom, preferably having between 4-7 carbon atoms, most preferably between 4-5 carbon atoms. It is preferred that such rings have between 1-3, and most preferably between 1-2 nitrogen heteroatoms.

In an even further preferred embodiment, in the method the invention said transition metal coupling reaction comprises reaction of the ¹⁸F-labelled synthon of Formula Y with the above-defined compound of Formula Ie in the presence of a source of carbon monoxide to obtain an ¹⁸F-labelled product of Formula IIf:

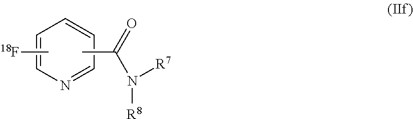

wherein R⁷ and R⁸ are as defined above for R⁵ and R⁶, respectively.

Example 3 relates to such a reaction.

The alternative known synthetic routes to form the above specific classes of compounds are relatively low-yielding as compared with the method of the present invention. For example, to obtain the compound of Formula IIf, one known method is via direct labelling, although this can be prohibitively low yielding in unactivated substrates. An alternative known method is a multi-stage activated ester strategy which is not straightforward to implement.

In a preferred embodiment, the method of the invention is automated, preferably on an automated synthesiser. [¹⁸F]-radiotracers are now often conveniently prepared on an automated radiosynthesis apparatus. There are several commercially-available examples of such apparatus, including Tracerlab™ and Fastlab™ (both from GE Healthcare Ltd). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. The present invention therefore provides in another aspect a cassette for carrying out the automated method of the invention wherein said cassette comprises:
i) a vessel containing a precursor compound of Formula X as defined above,
ii) means for eluting the vessel of step (i) with [¹⁸F]fluoride.

The cassette preferably also comprises:
iii) a vessel comprising a compound of any one of Formula Ia-e as defined above.

Where the cassette comprises the vessel comprising a compound of any one of Formula Ia-e, this vessel is eluted with the purified product of the reaction between the precursor compound of Formula X and [¹⁸F]fluoride, i.e. the synthon of Formula Y as defined herein. Purification is typically carried out by solid phase extraction on the cassette.

The cassette may also additionally comprise:
iv) an ion-exchange cartridge for removal of excess ¹⁸F.

### BRIEF DESCRIPTION OF THE EXAMPLES

Example 1 describes the Preparation of 2-Bromo-6[¹⁸F]-fluoropyridine.

Example 2 describes the preparation of 1-benzyl-4-(6-[¹⁸F]fluoropyridin-2-yl)piperazine.

Example 3 describes the preparation of N-benzyl-6-[¹⁸F]fluoropicolinamide.

Example 4 describes the preparation of 2-[¹⁸F]fluoro-6-(p-tolyl)pyridine.

Example 5 describes the preparation of 3-bromo-5-[¹⁸F]fluoropyridine.

Example 6 describes the preparation of 3-[¹⁸F]fluoro-5-(p-tolyl)pyridine.

### LIST OF ABBREVIATIONS USED IN THE EXAMPLES

Ac acetyl
BINAP 2,2′-bis(diphenylphosphino)-1,1′-binaphthalene
dba dibenzylideneacetone
DBU 1,8-diazabicyclo[5.4.0]undec-7-ene
DMF dimethylformamide
DMSO dimethylsulfoxide
HPLC high performance liquid chromatography
MeCN acetonitrile
NEt₃ triethylamine
Ph phenyl
THF tetrahydrofuran
UV ultraviolet

### EXAMPLES

### Example 1

### Preparation of 2-Bromo-6-[¹⁸]fluoropyridine

Experiments were undertaken to explore the optimum reaction conditions for preparing 2-bromo-6-[¹⁸F]fluoropyridine from 2,6-dibromo pyridine.

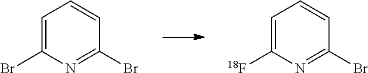 [SMILES: Brc1cccc(Br)n1 ; [18F]c1cccc(Br)n1]

All reactions were performed by conventional heating for ten minutes and the resulting 2-bromo-6-[¹⁸F]fluoropyridine was purified by semi-preparative HPLC using the following method:

### Column: ACE-5 C18 10×100 mm

MOBILE phase A=H₂O

### Mobile Phase B=MeCN

Flow rate 3 mL/min
Gradient 0-15 min, 5-95% B
In the table below, yields (from fluoride) are of the isolated product after HPLC purification, with yields in brackets being decay corrected.

| Quantity of | | Temperature/ | Yield | Synthesis |
|---|---|---|---|---|
| Br—Py—Br/mg | Solvent | ° C. | (d.c yield)/% | time/min |
| 5.0 | DMF | 120 | 42 (68) | 74 |
| 2.0 | DMF | 120 | 42 (64) | 65 |
| 2.0 | MeCN | 100 | 43 (67) | 70 |
| 2.0 | NMP | 100 | 18 (28) | 74 |
| 2.0 | DMF | 100 | 53 (79) | 64 |
| 2.0 | THF | 100 | 5 (7) | 68 |
| 1.0 | MeCN | 100 | 40 (60) | 67 |

The reaction highlighted in bold in the above table resulted in the highest yield.

### Example 2

### Preparation of 11-benzyl-4-(6-[¹⁸F]fluoropyridin-2-yl)piperazine

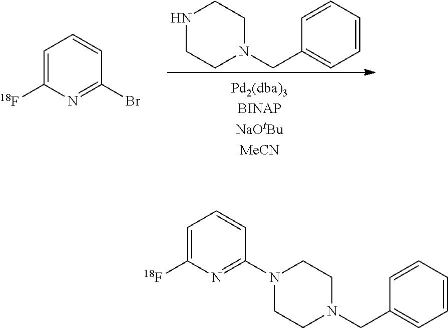 [SMILES: [18F]c1cccc(Br)n1 ; c1ccc(CN2CCNCC2)cc1 ; [18F]c1cccc(N2CCN(Cc3ccccc3)CC2)n1]

A Buchwald-Hartwig coupling reaction was tested with 1-benzylpiperazine, using tris(dibenzylideneacetone) dipalladium(0) and (±)BINAP with sodium t-butoxide in MeCN. After 25 min heating at 100° C., 49% of the activity was the desired product. The analytical HPLC using the following method:

### Column: Phenomenex Luna C18(2) 3μ 4.6×50 mm

Mobile phase A=0.8% NEt3 in H₂O, corrected to pH ˜7.5 with H₃PO₄
Mobile phase B=MeCN
Flow rate=1 mL/min

### 0-15 min 40-95% B

### 15-18 min 95% B

### 18-19 min 95-40% B

### 19-20 min 40% B

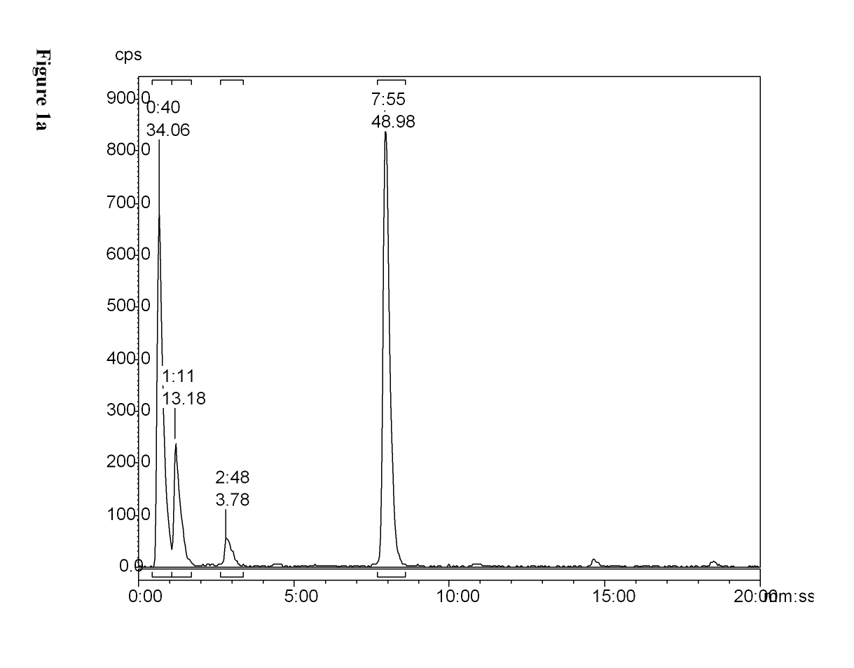

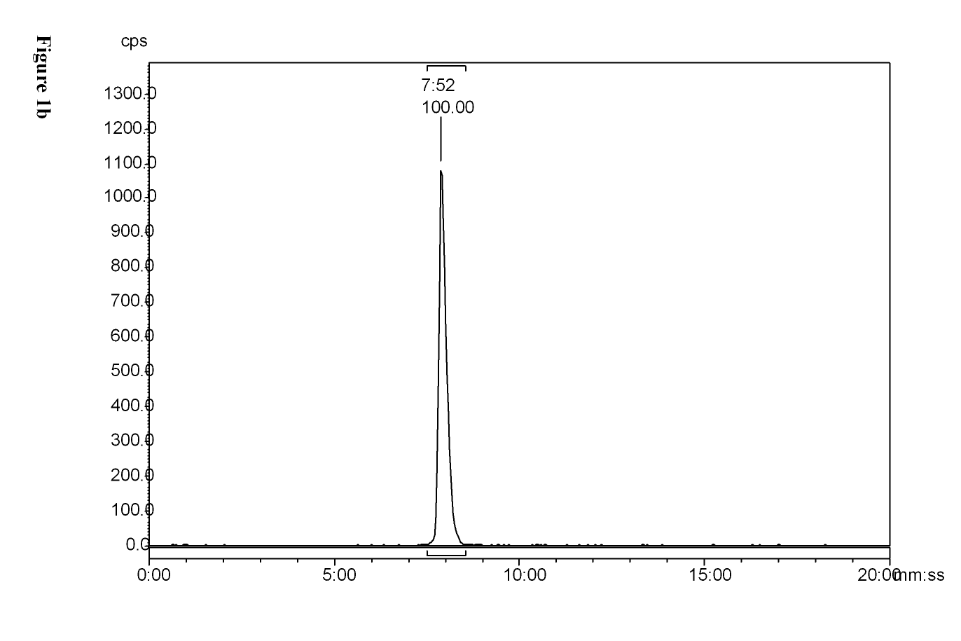

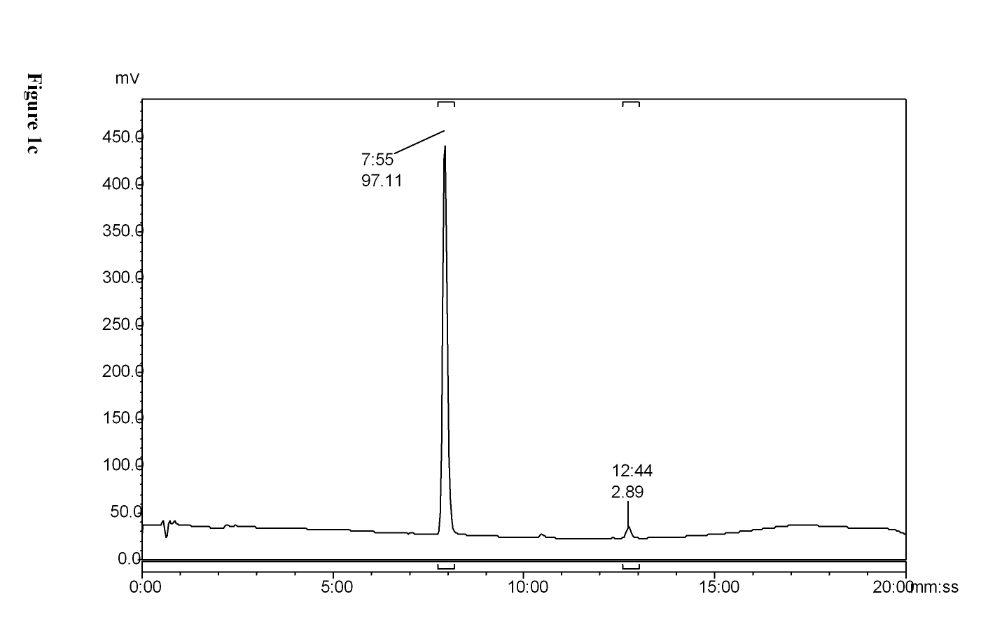

The traces are displayed in FIGS. 1a-c. FIG. 1a is a Radio-HPLC of the reaction mixture after 25 min heating at 100° C. FIG. 1b is a Radio-HPLC of the product after semi-preparative HPLC purification. FIG. 1c is a UV-HPLC (254 nm) of the [¹⁹F]standard.

### Example 3

### Preparation of N-benzyl-6-[¹⁸F]fluoropicolinamide

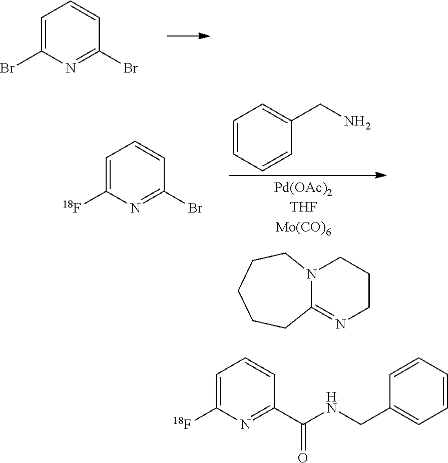

A reaction using molybdenum(0) hexacarbonyl as CO source was performed. In a procedure based on that described by Wannberg et al (2003 J Org Chem; 68: 5750), palladium acetate, molybdenum hexacarbonyl, benzylamine and 1,8-diazabicyclo[5.4.0]undec-7-ene (DBU) were added and the reaction was heated at 100° C.

Analytical HPLC was carried out using the following method:

### Column: Phenomenex Luna C18(2) 3μ 4.6×50 mm

Mobile phase A=0.8% NEt₃ in H₂O, corrected to pH ˜7.5 with H₃PO₄
Mobile phase B=MeCN
Flow rate=1 mL/min

### 0-15 min 40-95% B

### 15-18 min 95% B

### 18-19 min 95-40% B

### 19-20 min 40% B

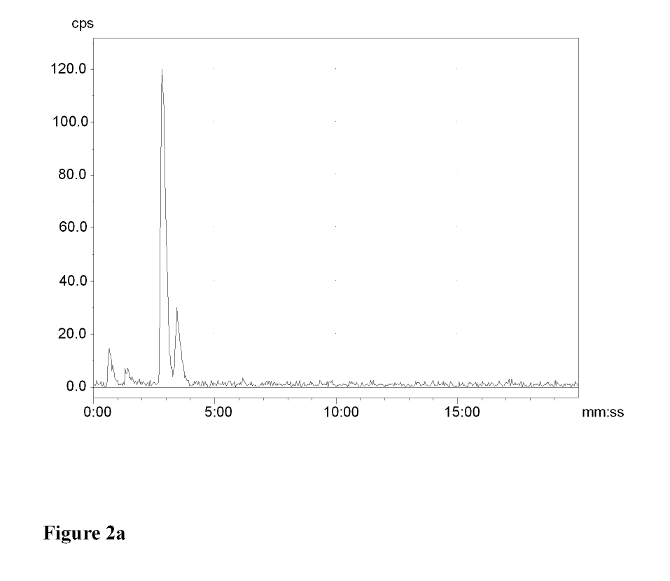

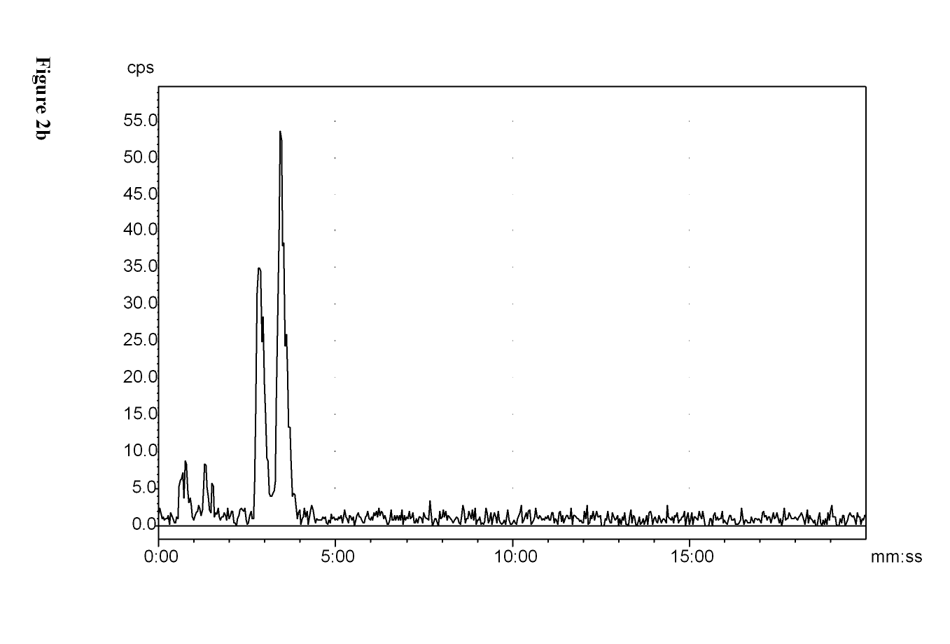

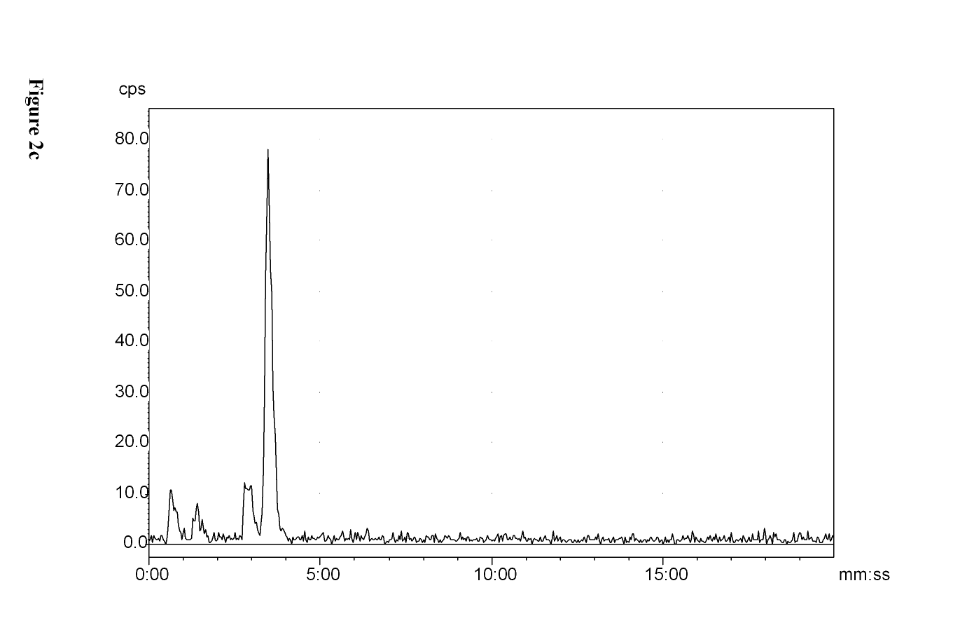

Traces of the aminocarbonylation reaction after 5, 15, and 30 min heating are displayed in FIGS. 2a-c, respectively. The retention times of [¹⁸F]-2-bromo-6-fluoropyridine and the desired product are 2.8 min and 3.5 min respectively. 66% of the activity injected was desired product after 30 min.

Analytical HPLC data a. Radio-HPLC 5 min, b. Radio-HPLC 15 min c. Radio-HPLC 30 min d. UV-HPLC of cold standard.

The absence of any [¹⁸F]-Buchwald-Hartwig product in the radiolabelling reaction (formed without insertion of CO) can be noted, demonstrating the efficiency of the aminocarbonylation reaction.

### Example 4

### Preparation of 2-[¹⁸F]fluoro-6-(p-tolyl)pyridine

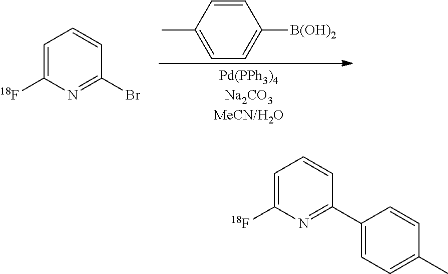

A Suzuki coupling was performed with p-tolylboronic acid, using tetrakis (triphenylphosphino) palladium and sodium carbonate in H₂O-acetonitrile mixture. After 5 min heating at 100° C., 98% of the activity was the desired product. Analytical HPLC was carried out using the following method:

### Column: Phenomenex Luna C18(2) 3μ 4.6×50 mm

Mobile phase A=0.8% NEt₃ in H₂O, corrected to pH ˜7.5 with H₃PO₄
Mobile phase B=MeCN
Flow rate=1 mL/min

### 0-15 min 40-95% B

### 15-18 min 95% B

### 18-19 min 95-40% B

### 19-20 min 40% B

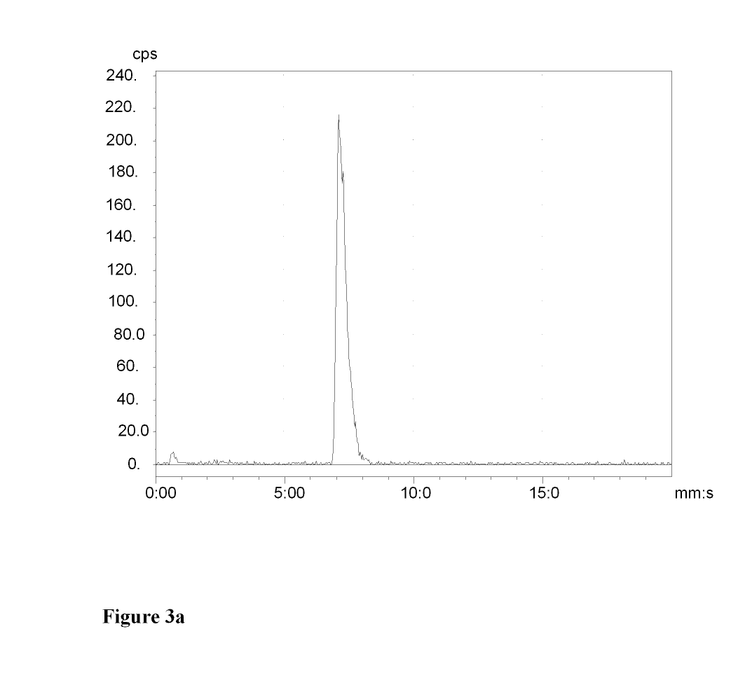

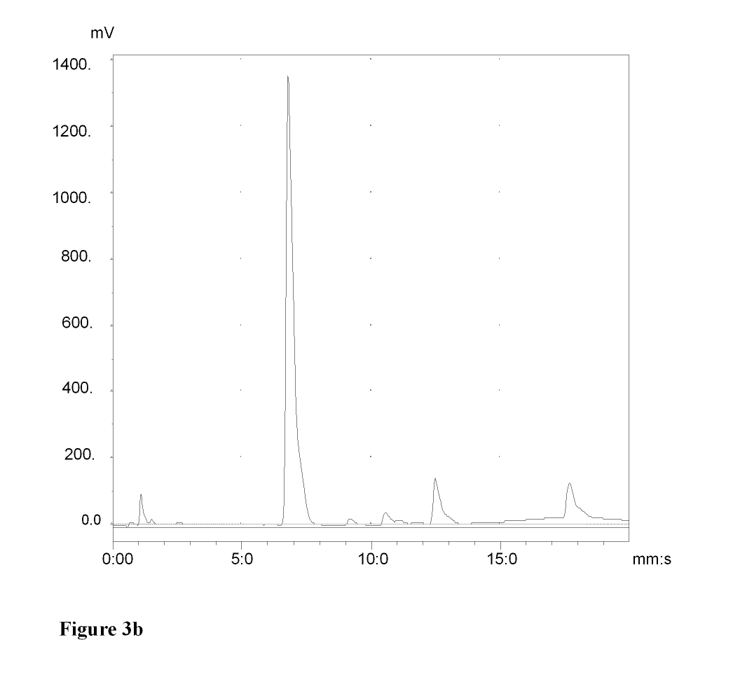

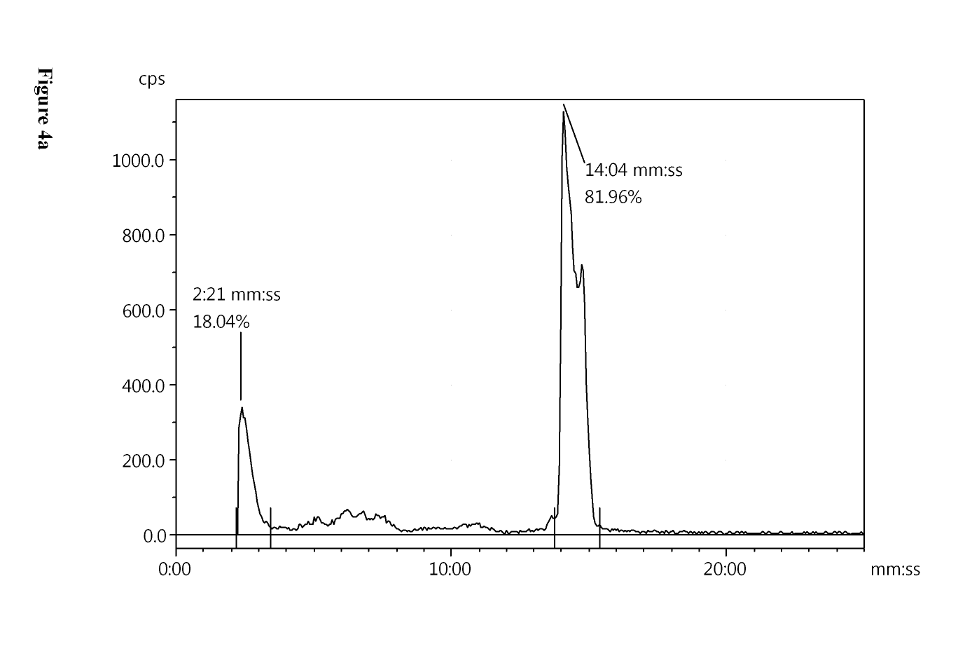

HPLC traces of the unpurified reaction is shown in the FIGS. 3a-b. FIG. 3a shows a Radio HPLC trace of the reaction after 5 min heating at 100° C. FIG. 3b shows a UV HPLC (254 nm) of the [¹⁹F]standard.

### Example 5

### Preparation of 3-bromo-5-[¹⁸F]fluoropyridine

Experiments to assess the [¹⁸F]fluorine labelling in the 3-position were undertaken.

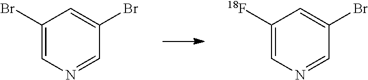 [SMILES: Brc1cncc(Br)c1 ; [18F]c1cncc(Br)c1]

Several experiments to explore various reaction conditions were performed. Yields given are after HPLC purification of the synthon (with the exception of entry 1). The HPLC method was as follows:

### Column: ACE5 C18 10×100 mm

Mobile phase A=H₂O

### Mobile Phase B=MeCN

Flow rate 3 mL/min

### 0-15 min 5-95% B

| Quantity of | | | | | |
|---|---|---|---|---|---|
| Br—Py—Br/ | Solvent | Temperature/ | Reaction | Yield (d.c | Synthesis |
| mg | (mL) | ° C. | time/min | yield)/% | time/min |
| 2.0 | MeCN | 100 | 10/20/30 | Analytical | n/a |
| | (0.2) | | | 2/2/2* | |
| 3.0 | DMF | Microwave | 1 | 6 (10) | 77 |
| | (0.2) | 100 | | | |
| 3.0 | DMSO | Microwave | 1 | 16 (24) | 64 |
| | (0.2) | 100 | | | |
| 5.0 | DMSO | 150 | 30 | 11 (20) | 90 |
| | (0.2) | | | | |
| 5.0 | DMSO | Microwave | 1 | 14 (19) | 55 |
| | (0.2) | 150 | | | |

*This is the analytical incorporation, by HPLC.

The highest yielding reaction was performed as follows:

3,5-dibromopyridine (3.0 mg) was added to dried [¹⁸F]fluoride/kryptofix/potassium carbonate in DMSO and subjected to microwave heating (50 W) for 1 min. After purification by semi-preparative HPLC, the isolated non-decay corrected yield from fluoride was 16%.

### Example 6

### Preparation of 3-[¹⁸F]fluoro-5-(p-tolyl)pyridine

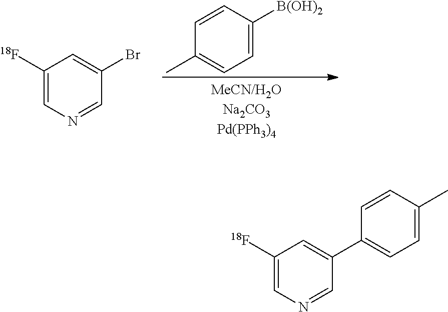

Suzuki coupling of 3-bromo-5[¹⁸F]fluoropyridine was performed with p-tolylboronic acid, using tetrakis(triphenylphosphino) palladium and sodium carbonate in an acetonitrile-H₂O mixture. After 5 min heating at 100° C., 82% of the activity was the desired product.

Semi-preparative radio-HPLC was carried out as follows:

### Column ACES C18 10×100 mm

### A=H₂O

### B=MeCN

Flow rate 3 mL/min

### 0-15 min 5-95% B

Analytical HPLC was carried out as follows:

### Column: Phenomenex Luna C18(2) 3μ 4.6×50 mm

Mobile phase A=0.8% NEt₃ in H₂O, corrected to pH ˜7.5 with H₃PO₄
Mobile phase B=MeCN
Flow rate=1 mL/min

### 0-15 min 40-95% B

### 15-18 min 95% B

### 18-19 min 95-40% B

### 19-20 min 40% B

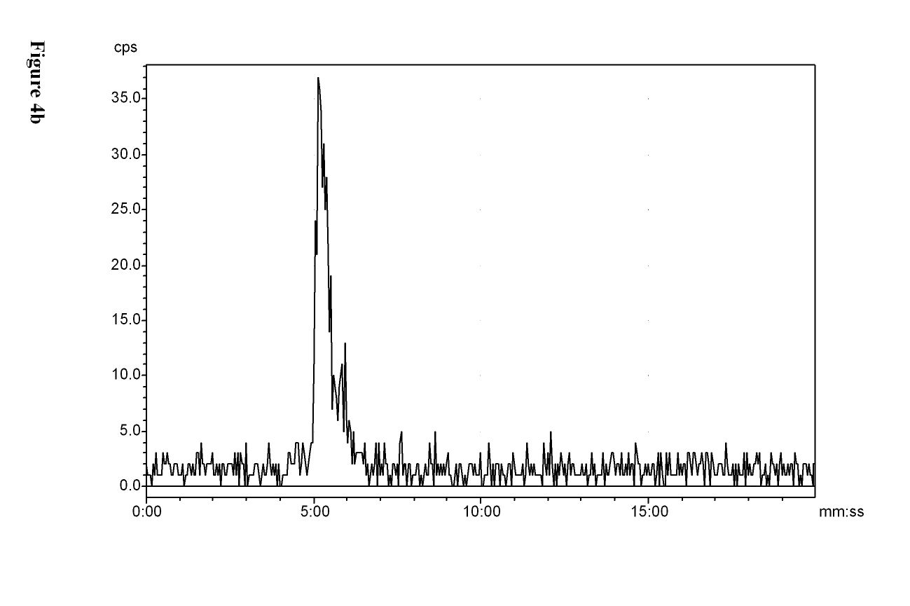

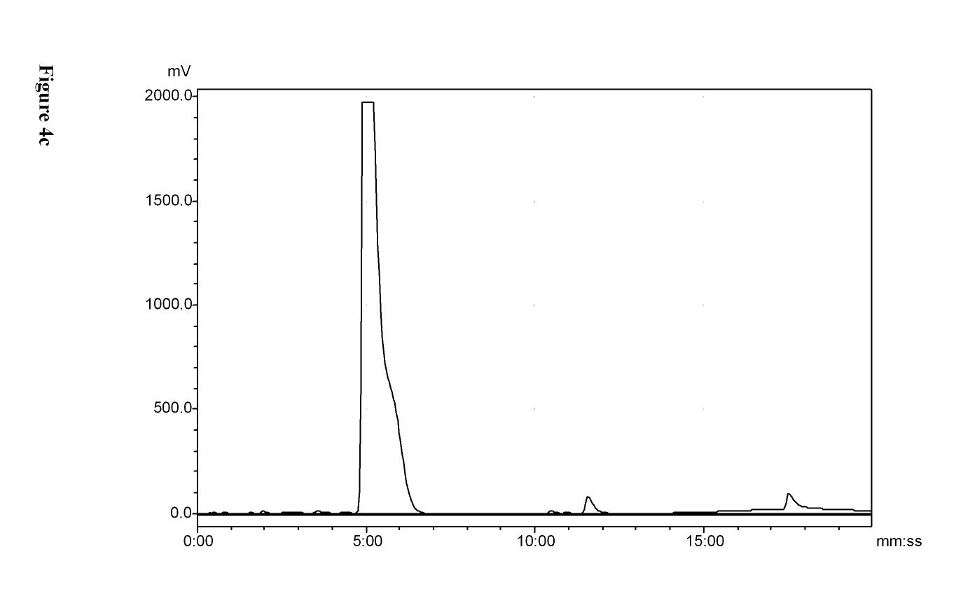

FIG. 4a shows the semi-preparative radio-HPLC trace of the Suzuki coupling reaction of p-tolylboronic acid and 3-bromo-5-[¹⁸F]fluoropyridine after 5 min at 100° C. Rₜ desired product=14.1 min. FIGS. 4b and 4c show the analytical HPLC traces of isolated 3-[¹⁸F]fluoro-5-(p-tolyl)pyridine. a. Radio-HPLC of isolated product. b. UV-HPLC of [¹⁹F]standard (254 nm). Rt product=5 min. The slight shoulder is due to the age of the column.

What is claimed is:

1) A method for [¹⁸F] labelling synthesis comprising reacting a radiolabelling precursor of Formula X: 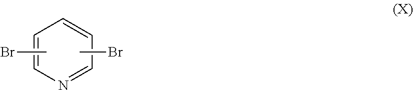
with [¹⁸F]fluoride to obtain an ¹⁸F-labelled synthon of Formula Y:

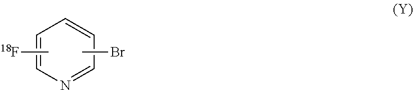

2) The method as defined in claim 1 wherein
said radiolabelling precursor of Formula X has the following chemical structure: 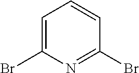
and said ¹⁸F-labelled synthon of Formula Y has the following chemical structure:

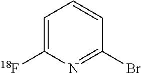

3) The method as defined in claim 1 wherein
said radiolabelling precursor of Formula X has the following chemical structure: 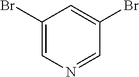
and said ¹⁸F-labelled synthon of Formula Y has the following chemical structure:

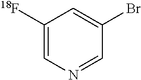

4) The method as defined in claim 1 which further comprises the step:
(ii) coupling the ¹⁸F-labelled synthon of Formula Y as defined herein with a cross-coupling partner in a transition metal-mediated coupling reaction to obtain an ¹⁸F-labelled product.

5) The method as defined in claim 4 wherein said transition metal is palladium.

6) The method as defined in claim 4 wherein said coupling step comprises reaction of said ¹⁸F-labelled synthon of Formula Y with a compound of Formula Ia: 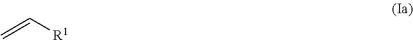
wherein R¹ is a monovalent aliphatic or aromatic hydrocarbon group;
to obtain an ¹⁸F-labelled product of Formula IIa:

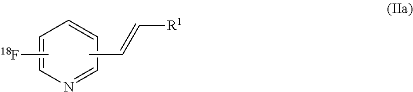

7) The method as defined in claim 4 wherein said coupling step comprises reaction of the ¹⁸F-labelled synthon of Formula I with a compound of Formula Ib:
Bu₃SnR² (Ib)
wherein R² is a monovalent aliphatic or aromatic hydrocarbon group;
to obtain an ¹⁸F-labelled product of Formula IIb:

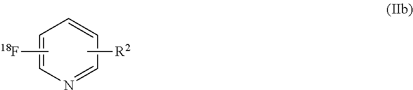

8) The method as defined in claim 4 wherein said coupling step comprises reaction of the ¹⁸F-labelled synthon of Formula I with a compound of Formula Ic: 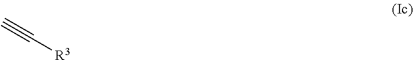
wherein R³ is a monovalent aliphatic or aromatic hydrocarbon group;
to obtain an ¹⁸F-labelled product of Formula IIa:

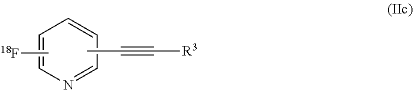

9) The method as defined in claim 4 wherein said coupling step comprises reaction of the ¹⁸F-labelled synthon of Formula I with a compound of Formula Id: 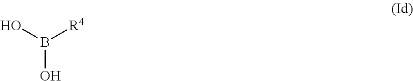
wherein R⁴ is a monovalent aliphatic or aromatic hydrocarbon group;
to obtain an ¹⁸F-labelled product of Formula IId:

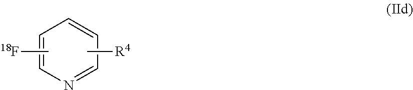

10) The method as defined in claim 4 wherein said coupling step comprises reaction of the ¹⁸F-labelled synthon of Formula I with a compound of Formula Ie: 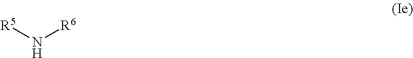
wherein R⁵ and R⁶ are independently hydrogen or a monovalent aliphatic or aromatic hydrocarbon group, or together with the nitrogen to which they are attached form a nitrogen-containing aliphatic or aromatic ring;
to obtain an ¹⁸F-labelled product of Formula IIe:

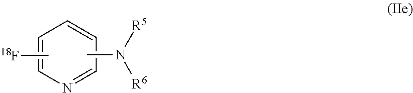

11) The method as defined in claim 4 wherein said coupling step comprises reaction of the ¹⁸F-labelled synthon of Formula I with a compound of Formula Ie 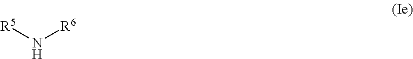
wherein R⁵ and R⁶ are independently hydrogen or a monovalent aliphatic or aromatic hydrocarbon group, or together with the nitrogen to which they are attached form a nitrogen-containing aliphatic or aromatic ring;
in the presence of a source of carbon monoxide to obtain an ¹⁸F-labelled product of Formula IIf: 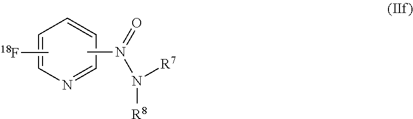
wherein R⁷ and R⁸ are as defined for R⁵ and R⁶, respectively.

12) The method as defined in claim 1 which is automated.

13) A cassette for carrying out the method as defined in claim 12 wherein said cassette comprises:
i) a vessel containing a precursor compound of Formula X 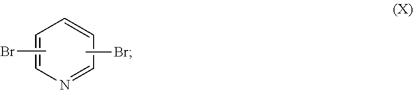
ii) means for eluting the vessel of step (i) with [¹⁸F]fluoride.

14) The cassette as defined in claim 13 which further comprises:
iii) a vessel comprising a compound of any one of Formula Ia-e 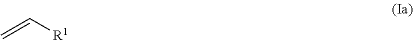
wherein R¹ is a monovalent aliphatic or aromatic hydrocarbon group;
Bu₃SnR² (Ib)
wherein R² is a monovalent aliphatic or aromatic hydrocarbon group; 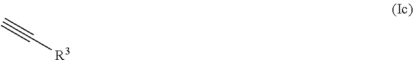
wherein R³ is a monovalent aliphatic or aromatic hydrocarbon group; 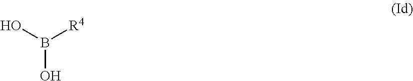
wherein R⁴ is a monovalent aliphatic or aromatic hydrocarbon group; 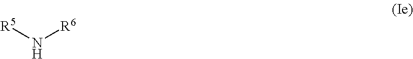
wherein R⁵ and R⁶ are independently hydrogen or a monovalent aliphatic or aromatic hydrocarbon group, or together with the nitrogen to which they are attached form a nitrogen-containing aliphatic or aromatic ring.
